(19) United States
(12) Patent Application Publication
(10) Pub. No.: US 2026/0315039 A1
(43) Pub. Date: Oct. 15, 2026

(54) DETERMINING SUB-NOISE GAIN LEVEL FOR ULTRASOUND

(71) Applicant: NEWSOUTH INNOVATIONS PTY LIMITED, Sydney, New South Wales (AU)

(72) Inventors: Alec W WELSH, Sydney, New South Wales (AU); Gordon STEVENSON, Sydney, New South Wales (AU)

(21) Appl. No.: 18/998,791

(22) PCT Filed: Jul. 27, 2022
(86) PCT No.: PCT/AU2022/050787

§ 371 Date: Jan. 27, 2025

(51) Int. Cl. A61B 8/00

(52) U.S. Cl. CPC A61B 8/5269; A61B 8/461; A61B 8/488; A61B 8/54

(57) ABSTRACT

A machine readable storage medium stores instructions for determining a sub-noise gain level for an ultrasound scan of a tissue volume of interest. A series of ultrasound data sets are received, each ultrasound data set corresponding to a same tissue volume of interest at a different respective power Doppler gain level. A power Doppler data percentage is determined for each ultrasound data set to form a series of power Doppler data percentages. Statistical analysis is applied to the series of power Doppler data percentages to determine a breakpoint which corresponds with a noise bloom and a sub-noise gain level is determined based upon the breakpoint. An ultrasound apparatus can be used to carry out a method of implementing the instructions.

### TECHNICAL FIELD

This disclosure relates to a method and system for determining a sub noise gain level for an ultrasound scan. It may for example be applied to a power Doppler ultrasound machine to select appropriate settings for imaging a tissue volume of interest and/or appropriate settings for functional measurement.

### BACKGROUND ART

Medical imaging is commonly used in modern health care management and diagnostics. Ultrasound has clear potential advantages as an inexpensive tool currently used for structural evaluation. Ultrasound imaging can be performed in 2D or 3D and is used for diagnosis and monitoring of diseases and pathology across medicine. It can provide images of organ/tissue structure and function non-invasively, cheaply and at the bedside. An example is the application of ultrasound imaging to diagnose tumour, as tumour tissue is considered to have higher perfusion. Perfusion of an organ or tissue volume of interest refers to the rate of blood flow to the organ or tissue volume of interest. For example, perfusion may be measured as the volume of blood delivered to the organ or tissue volume of interest per unit volume of tissue per unit of time.

Brightness mode (B-mode) ultrasound refers to ultrasound imaging in which the organs and tissues of interest are depicted by points of variable brightness. The brightness of each point is determined by the amplitude of the pulse echo. In addition to B-Mode ultrasound, modern ultrasound machines can perform Doppler ultrasound which uses the Doppler effect to image the movement of tissues and body fluids (such blood). In this way substantial information relating to tissue or organ function can be generated by using Doppler modes (frequency or phase shift evaluation) to estimate blood flow. For example, functional measures may be performed by Doppler ultrasound which uses the phase shift of ultrasound pulses to provide a measurement of the amount of blood flow present in a region of interest.

Ultrasound data may be acquired using a transducer. The transducer may scan in a single plane line-by-line across an area of tissue where each individual vertical scan line is transmitted and echoes are received from differing depths. These multiple lines may be summated to create a two-dimensional image of the tissue or area of insonation beneath the ultrasound transducer. An extension of this, is 3D ultrasound imaging. In 3D ultrasound, a motorized transducer, such as but not limited to a curvi linear transducer, may be used to sweep through a volume recording multiple two dimensional (2D) sweeps to form a full 3D volume.

Ultrasound machines may interrogate individual vessels, tumours, whole vascular beds or pathology with differences in perfusion. The display of this information is typically in two-or three-dimensions as a colour map indicating information relating to either the mean velocity of blood flow (colour Doppler) or the amplitude of the Doppler signal which is proportional to the amount of moving blood (power Doppler). Power Doppler indicates the energy and not the direction of flow, and is more sensitive to low rates of flow than colour Doppler. Therefore power Doppler is particularly suitable for examining tumours, tiny low-flow vessels, and subtle ischemic areas etc.

Sources of error in perfusion quantification and other measurements using ultrasound include signal attenuation due to depth, patient habitus and tissue inhomogeneity, which reduces the possibility for comparison over time or between patients.

To compensate for differences between patients, and differences in the same tissue at different scan-times, a technique called Fractional Moving Blood Volume (FMBV) has been proposed. FMBV allows standardisation within an image or volume of tissue relative to an adjacent region of known vascular amplitude (such as a large local blood vessel). The known vascular amplitude is chosen as the 100% value, against which the measurement is compared. That is, the known vascularity of a large artery is chosen as an internal standardisation point (i.e. 100% flow). It is used as an internal reference from which to relatively measure vascularity. Once the reference point is chosen, the power Doppler (PD) ultrasound is performed to scan across the tissue.

The gain level (“power Doppler gain level”) at which power Doppler ultrasound data is acquired refers to the level of amplification applied to the raw ultrasound data. When performing a power Doppler ultrasound scan it is desirable to set the power Doppler gain level to a level which maximises display of vascular information, but which is not so high as to introduce high levels of noise which may obscure the image. Appropriate levels are thus referred to as sub-noise gain (SNG) levels, as they are below a level at which the image and underlying data is obscured by gain-induced noise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

A first aspect of the present disclosure provides a method for determining a sub-noise gain level for performing a power Doppler ultrasound scan of a tissue volume of interest. The method comprises:
receiving a series of ultrasound data sets, each ultra sound data set corresponding to a same tissue volume of interest, each ultrasound data set having been acquired at a different respective power Doppler gain level;
for each ultrasound data set, determining a power Doppler data percentage, being a percentage of data in the ultrasound data set which corresponds to power Doppler data, wherein the determined power Doppler data percentages form a series of power Doppler data percentages corresponding to the series of ultrasound data sets;
applying statistical analysis to the series of power Doppler data percentages, to determine a breakpoint in the series of power Doppler data percentages which corresponds with a noise bloom; and
determining a sub-noise gain level based upon a power Doppler gain level corresponding to the breakpoint or based upon a power Doppler gain level corresponding to a point calculated from the breakpoint.

In some examples, determining the sub-noise gain level may comprise looking up a power Doppler gain level at which an ultrasound data set corresponding to the breakpoint was acquired. In other examples, determining the sub-noise gain level comprises determining a power Doppler gain level of a point calculated from the breakpoint. The point calculated from the breakpoint may be offset by a predetermined amount (e.g. a predetermined number of frames or period of time) from the breakpoint.

The method may comprise filtering the power Doppler data percentages with a rolling window. The filtering with a rolling window may comprise averaging the power Doppler data percentages over the rolling window.

In some examples, the ultrasound data may be provided in a format that is native to the ultrasound machine and not displayed, but contains the information of Doppler data and gain values. Where native format data is not available, the analysis may be performed on an image displayed or output by the ultrasound machine.

In some examples, each ultrasound data set includes an ultrasound image comprising an ultrasound cone portion, and the percentage Doppler data percentage for each ultrasound data set may be determined by segmenting pixels in the ultrasound cone portion of the image which have power Doppler data values. In some examples, the ultrasound image further includes a power Doppler colour bar portion, wherein the segmented pixels have colour values which are also found in any of the pixels in the power Doppler colour bar portion.

The statistical analysis may comprise determining an inflection point in the series of power Doppler percentage data. In some examples, the statistical analysis comprises a piece-wise separation of the series of power Doppler percentage data into two or more regression models. The regression models may be based on a known (e.g. predetermined) number of segments.

In some examples, the method comprises initialising an ultrasound machine by controlling the ultrasound machine to generate the series of power Doppler ultrasound data sets by performing a plurality of power Doppler ultrasound scans at a plurality of different power Doppler gain levels at a same tissue volume of interest.

In some examples, the method comprises determining the sub-noise gain level for the tissue volume of interest and then performing a power Doppler ultrasound scan of the tissue volume of interest at the determined sub-noise gain level.

In some examples, the tissue volume of interest may be divided into a plurality of sub-volumes of interest based on depth, wherein each sub-volume of interest corresponds to a different depth. The method may comprise scanning each sub-volume of interest at the sub-noise gain level determined for said sub-volume of interest. This makes it possible to scan a tissue volume of interest at multiple depths and to apply an appropriate sub noise gain level to each depth.

A second aspect of the present disclosure provides a non-transitory machine readable storage medium storing instructions executable by a processor to implement the method of the first aspect of the present disclosure.

A third aspect of the present disclosure provides an ultrasound system configured to perform the method of the first aspect of the present disclosure.

A fourth aspect of the present disclosure provides an ultrasound apparatus comprising:
a transducer for sending and receiving an ultrasound signal;
a display for displaying ultrasound data;
a controller for controlling the transducer to perform a power Doppler ultrasound scan; and
a sub-noise gain module configured to determine a sub-noise gain level for performing power Doppler ultrasound on a tissue volume of interest in accordance with the method of the first aspect of the present disclosure; wherein the sub-noise gain module is implemented by a processor.

Any of the features of the first to fourth aspects of the present disclosure discussed above may be combined with each other.

### BRIEF DESCRIPTION OF THE DRAWINGS

Embodiments will now be described by way of example only, with reference to the accompanying drawings in which

FIG. 1A is a scatter graph showing the attenuation of the ultrasound wave for water and different tissues, expressed in decibels against the distance from the transducer head;

FIG. 1B is a scatter graph showing the attenuation data in FIG. 1A, but expressed as a percentage of change in the ultrasound level, with the ultrasound level at 0 cm distance from the transducer head as the reference value (100%);

FIG. 2A depicts power Doppler imaging data in which an organ of interest is annotated;

FIG. 2B depicts an organ image segmented from FIG. 2A, further annotated by colour to show the sampling of the organ into slices with different depths;

FIG. 2C shows the organ image shown in FIG. 2B, but segmented with a single mask as a single region of interest;

FIG. 3 schematically depicts an ultrasound machine including a module for determining a sub-noise gain level according to one example of the present disclosure;

FIG. 4 schematically depicts a method for determining a sub-noise gain level according to one example of the present disclosure;

FIG. 5 schematically depicts another method for determining a sub-noise gain level, according to one example of the present disclosure;

FIG. 6 shows plots of power Doppler data percentages and power Doppler gain levels as a function of position in a series of ultrasound data sets according to one example of the present disclosure; and

FIGS. 7A to 7D depict a series of ultrasound images combing B-mode image and power Doppler image, at different sub-noise gain levels.

FIGS. 8A to 8D are line drawings depicting the series of ultrasound images of FIGS. 7A to 7D.

### DETAILED DESCRIPTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

As used herein, the term ‘image’ broadly refers to a viewable image or data representing a viewable image. An image may, for example, be generated by an ultrasound mode such as, but not limited to, B-mode, CF-mode and/or sub-modes of CF such as power Doppler (PD), tissue velocity imaging (TVI), Angio, B-Flow, MM, CM, pulsed wave (PW), tissue velocity Doppler (TVD) and continuous wave (CW). The image may, for example, be generated by a single ultrasound beam or multiple ultrasound beams.

Various embodiments may employ processors or processing units to process data. Examples of processors or processing units include, but are not limited to, central processing units (CPU), graphic processing units (GPU), digital signal processors, field-programmable gate arrays (FPGA), application-specific integrated circuits (ASIC) etc. and/or combinations thereof. The processor or processing unit may execute machine readable instructions stored in a memory or computer readable storage medium to implement the methods described in the examples and embodiments herein.

As discussed above, power Doppler ultrasound is an imaging technique which may be used to produce images of a tissue volume of interest. For example, the power Doppler data may be used to gather data on the rate of flow of fluid (e.g. blood) and thus used to map areas of interest.

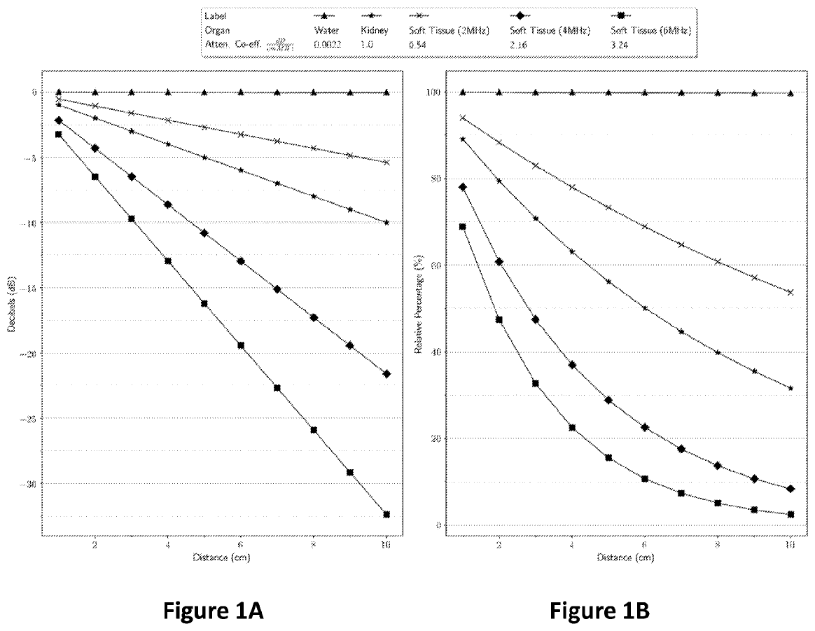

One challenge is that, due to attenuation, power Doppler readings are sensitive to the depth to the target. FIGS. 1A and 1B show the relationship between attenuation and depth from the transducer head based on theoretical transmission of ultrasound through mediums with different density or attenuation profiles and frequency. As a result, if the distance from the transducer to the target varies across patients, or varies with different readings on the same patient, with no internal standardization, this can result in inaccurate perfusion measurements.

As discussed above, Fractional Moving Blood Volume (FMBV) is a technique used in ultrasound which allows standardisation within an image or volume of tissue relative to an adjacent region of known vascular amplitude (such as a large local vessel). FMBV may, for example, be applied in 3D ultrasound to generate a whole organ measurement of perfusion using power Doppler ultrasound.

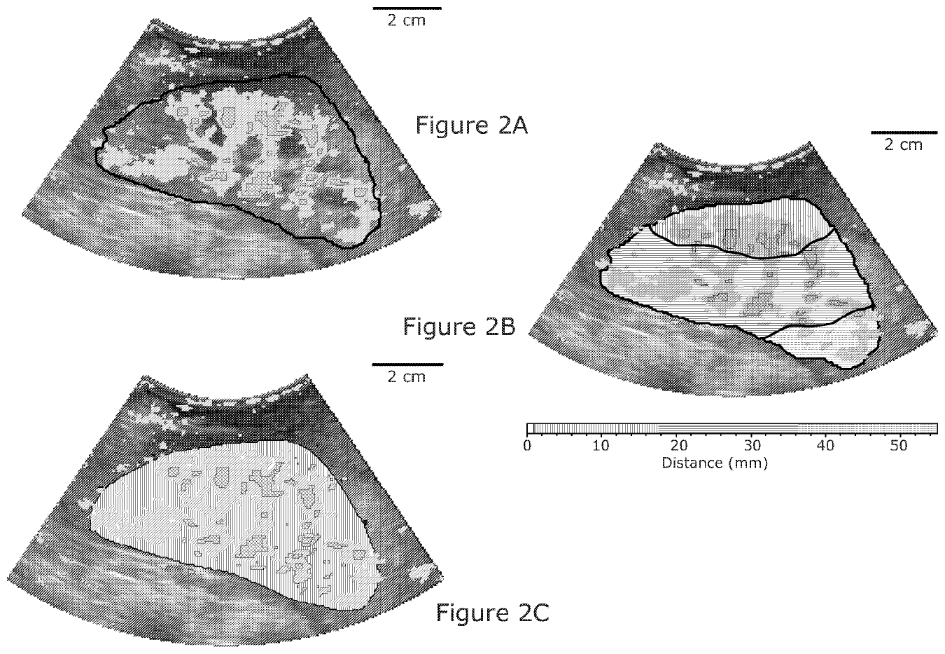

An example of imaging data acquired using FMBV and 3D ultrasound is shown in FIG. 2A. In FIG. 2A, a tissue volume of interest, in this case an organ, has been annotated based on the ultrasound data. FIG. 2B shows the organ image of FIG. 2A, but further annotated by color to show the sampling of the organ into slices with different depths. FIG. 2C shows the organ image shown in FIG. 1B, but segmented or labelled as a single region of interest. In all of these images, useful information about the region of interest can be derived from the power Doppler data which is shown in color in FIG. 2A.

Another challenge is that power Doppler ultrasound data is very sensitive to the gain level used by the ultrasound machine. This is the case for all types of power Doppler ultrasound data and may be particularly serious when using power Doppler ultrasound for perfusion estimation or FBMV on tissue containing small blood vessels.

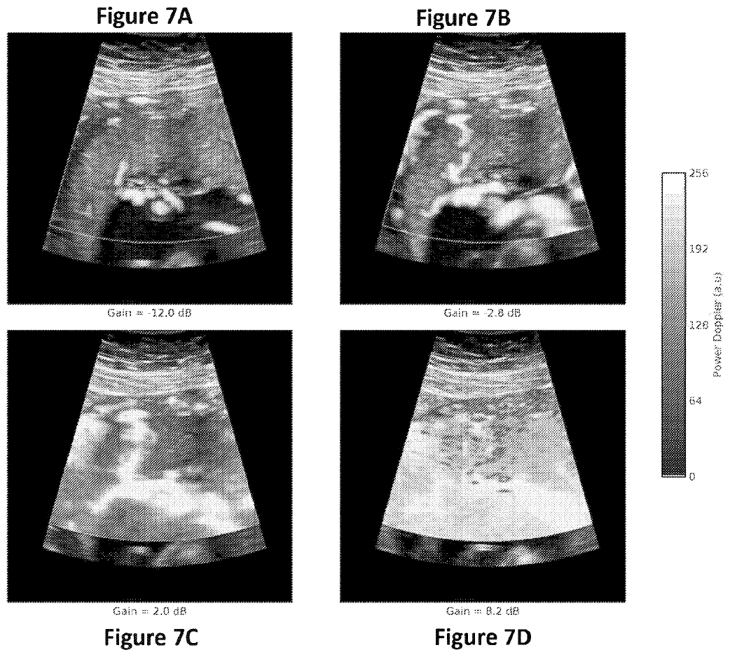

FIGS. 7A to 7D depict the effect of the gain level on the resulting scan data.

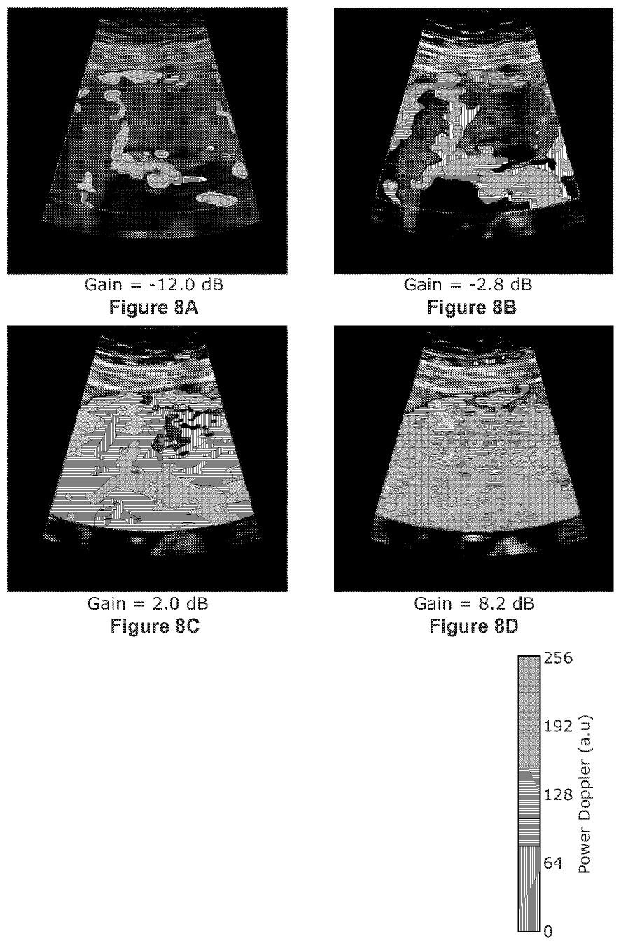

FIGS. 8A to 8D correspond to FIGS. 7A to 7D and show the same scan data, but using line drawings with hatched shading for clarity. As shown in FIG. 7A (and FIG. 8A), at a −12.0 dB gain level, the power Doppler data is insufficiently amplified. In contrast, FIG. 7B (and FIG. 8B), shows the power Doppler data obtained at a −2.8 dB gain level, which in this case is an appropriate gain level with adequate amplification. However, when the gain level is increased to 2.0 dB, as shown in FIG. 7C, the higher gain produces an artefact in the power Doppler data where scan noise has been amplified to the extent that it obscures the functional data. The artefact in the power Doppler image of FIG. 7C (and FIG. 8C) presents a “bloom effect”, where a noise “bloom” in the power Doppler data can be seen in comparison to FIGS. 7A and 7B. That is in FIG. 7C, a substantial amount of data presents as “power Doppler” data even though it is in fact noise. In FIG. 7D (and FIG. 8D), the gain level is set at 8.2 dB. It can be seen that, at this gain level, the noise effect is amplified to such a degree that the demarcation between true power Doppler data and the noise in the scan is no longer possible. Thus, the gain levels of 2.0 dB and 8.2 dB shown in FIGS. 7C and 7D are too high and cannot be considered sub-noise gain levels. However, the gain levels of FIGS. 7A and 7B may be considered sub-noise gain levels.

Thus it will be appreciated that in power Doppler ultrasound, intensity information can be skewed by under or over saturation due to inappropriate settings of the power Doppler gain value. Overly high gain values can lead to a “bloom effect” where noise corrupts the vascular information, for instance as shown in FIG. 7D. However, with the gain set too low, the true vascular signal may not be displayed due to the cut-off effect of the high-pass wall motion filter, for instance as shown in FIG. 7A.

Selecting the appropriate gain level for power Doppler imaging is a difficult task. This is especially the case for FMBV, where there may be differences in depth and tissue density between patients resulting in attenuation or loss of signal and echo strength with depth. In prior art ultrasound image acquisition processes, the sub-noise gain (SNG) level was manually selected by the sonographer based on their experience and visual cues from the images. However, relying on the operator is subject to human error and may make it difficult to obtain reproducible results as different operators may make different choices based on their subjective evaluation. Furthermore, if an inappropriate gain level is chosen (too low or too high) this will negatively impact the acquired ultrasound data.

Accordingly, the present disclosure proposes an apparatus and method which can automatically determine an appropriate sub-noise gain level setting for a tissue volume of interest, based on ultrasound data acquired from a tissue volume of interest. The appropriate level may be referred to as an optimal “sub-noise gain level” (SNG), where the term “optimal” is to be understood in a general sense as meaning a level which amplifies the true signal to at least a detectable level, while avoiding a noise bloom.

Selecting the sub-noise gain level may, for example, be done as part of a set-up or initialisation process, where initial ultrasound data is acquired and processed to determine the appropriate SNG setting to be used during the subsequent scanning to obtain the actual data to be used for medical analysis. As the process of determining the SNG level according to the present disclosure is automated, this may reduce the training required for ultrasound operators and may improve reliability and reproducibility of ultrasound scans.

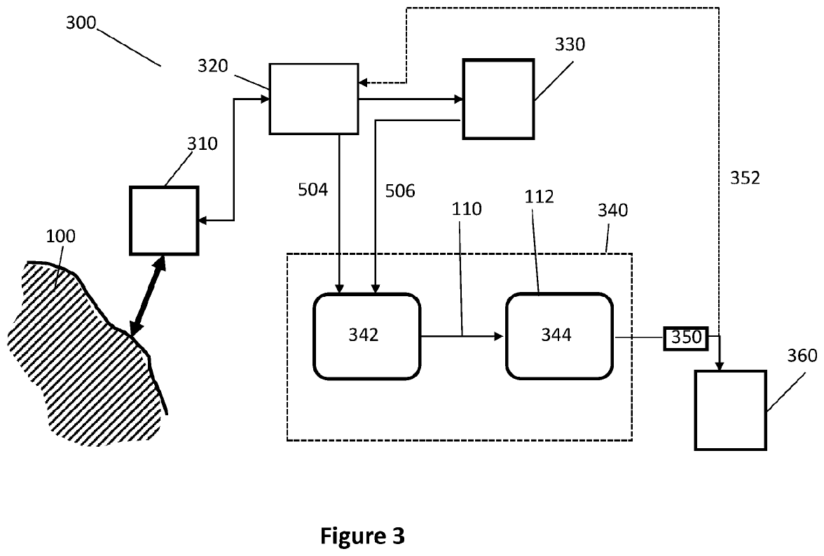

FIG. 3 is a schematic diagram depicting an ultrasound apparatus 300 according to one example of the present disclosure. The ultrasound apparatus 300 includes a transducer 310, a controller 320 and a display 330. The transducer 310 is configured for acquiring ultrasound data from the scanned tissue 100 and may transmit the acquired ultrasound data to a controller 320. The ultrasound data may be used to generate images for display on a screen or other display device 330. The controller 320 may be implemented by one or more processors and may execute instructions stored in a memory or machine readable storage medium.

The ultrasound machine 300 has a module 340 for determining a SNG level. The module 340 is configured to implement a method for determining an optimal SNG level. By automatically determining the optimal SNG level, it may be possible to obtain consistent, reproducible, high quality readings and avoid human error or variations due to subjective human judgement. This is in contrast to conventional methods in which the gain is set by the sonographer based on their subjective interpretation.

In some examples, the optimal gain level 350 determined by the module 340 may be provided or communicated to the sonographer. For example the determined optimal gain level 350 may be output on a display device 360 which receives an output from the module 340. The sonographer can then adjust the SNG setting of the apparatus 300 to the calculated optimal gain level shown on the display 360, and restart or resume the scanning process.

In other examples, the optimal gain level determined by the module 340 may instead be provided to the controller 320 of the ultrasound machine (as indicated by dashed arrow 352). The gain level of the ultrasound machine may then be automatically adjusted by the controller 320 of the ultrasound machine.

The module 340 may be implemented by a processor and/or by machine readable instructions stored in memory and executable by a processor. While module 340 is shown as a separate entity in FIG. 3, in other examples, the module 340 may be provided as part of the controller 320. For example, the module 340 may for example be implemented by machine readable instructions stored in memory and executable by a processor of the controller 320.

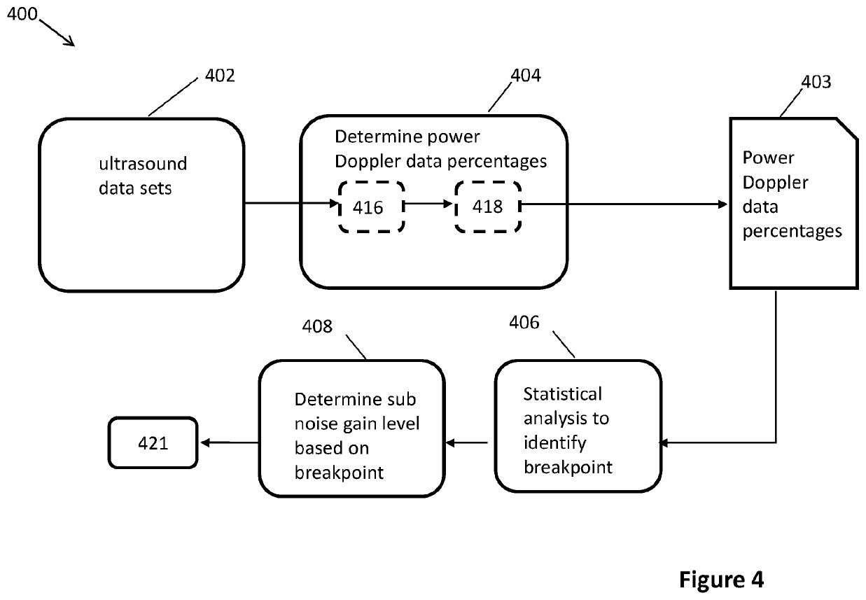

FIG. 4 schematically depicts one example of a method 400 that may be employed by the module 340 to determine the optimal sub-noise gain level for a volume of tissue of interest.

The method 400 processes acquired power Doppler ultrasound data in order to determine the appropriate sub-noise gain for a tissue volume of interest. In some examples, the method 400 may be applied to tissue volume of interest as a whole to find a single optimal sub-noise gain level to be used with the tissue volume of interest. In other examples, the tissue volume of interest may be split into a plurality of sub-volumes of interest based on depth and the sub-noise gain level may be calculated separately for each sub-volume of interest.

That is, in some implementations, the tissue volume of interest may be divided into different regions by depth, so each sub-volume of interest may correspond to the same area but at different depths. By re-calculating the sub-noise gain level for each sub-volume, an appropriate sub-noise gain level may be determined for each depth, as different pieces of tissue at different depths may have different optimal sub-noise gain levels. For instance, each sub-volume may span a fixed range of depths. For example, a first sub-volume may span depths from X −0.5 cm to X+0.5 cm, a second sub-volume X+0.5cm to X+1.5 cm, a third sub-volume may have a depth of X+1.5 cm to X+2.5 cm etc., where X is a depth of the shallowest scan.

At block 402 the method comprises receiving a series of ultrasound data sets, each ultra sound data set corresponding to a same tissue volume of interest (or the same sub-volume of interest, where the tissue volume of interest is divided into a plurality of sub-volumes). Each ultrasound data set may be acquired at a different respective power Doppler gain level. In this way a series of ultrasound data sets corresponding to a range of power Doppler gain levels is acquired.

Block 402 may be performed over a period of time, during which the power Doppler gain level is varied. For instance, the gain level may be increased from a minimum level at the start of the time period, to a maximum level at the end of the time period. In this way a number of image frames may be acquired and each image frame corresponds to an ultrasound data set acquired at a particular gain level. In other examples, the gain level may be reduced from an initially high level or otherwise varied, but in any case each ultrasound data set corresponds to a respective power Doppler gain level which is recorded together with the ultrasound data set.

The series of power Doppler ultrasound data sets may, for example, be captured using a commercial ultrasound machine and may comprise a sequential, chronological series of ultrasound data sets acquired at a frame rate determined by the ultrasound machine. During this process, the ultrasound machine may produce 2D image frames for display on a monitor.

Each image frame corresponds to an ultrasound data set and includes power Doppler data and other data, such as structural “B-mode” data. In FIGS. 7A-7D, which are representative images, the power Doppler data can be seen as the bright coloured parts of the image. As the gain level is gradually increased in block 402 while acquiring the ultrasound data sets, it can be expected that the amount of power Doppler data observed in the later image frames (later ultrasound data sets) will be larger than the amount power Doppler data observed in the earlier data sets.

At block 404 the method comprises, for each ultrasound data set, determining a power Doppler data percentage. The Doppler data percentage is a percentage of data in the ultrasound data set which corresponds to power Doppler data. The power Doppler data percentages determined by the module thus form a series of power Doppler data percentages corresponding to the series of ultrasound data sets. That is each determined power Doppler data percentage has a respective position in the series of power Doppler data percentages and corresponds to a respective ultrasound data set having a corresponding position in the series of ultrasound data sets.

Block 404 thus comprises determining, for each ultrasound data set, the amount of data which corresponds with power Doppler data. In some cases where the acquired ultrasound data sets 402 are a series of ultrasound images, the method involves determining, in each of the 2D ultrasound cone images, the percentage of the total image area that correspond to power Doppler data as opposed to the structural “B-mode” data. For instance, this may be calculated by dividing the number of pixels determined to have power Doppler data, by the total number of pixels. The total number of pixels here is the sum of the number of pixels determined to have power Doppler data, and the number pixels determined to contain B-mode data. These calculations result in a series of “power Doppler” percentage values 403, one for each image frame in the series of ultrasound image frames.

In some examples, determining the power Doppler data percentages may include calculating a power Doppler data percentage for each ultrasound data set individually 416 and then applying a filtering process 418 to smooth the data.

In some examples, the module 340 for determining the SNG may read the ultrasound data directly from a memory or storage medium of the ultrasound machine. In other examples, if the underlying ultrasound data cannot be accessed directly, image processing algorithms may be applied to images shown on the ultrasound machine's display 330 or to ultrasound images otherwise output from the ultrasound machine, in order to determine 416 the power Doppler data percentage values.

In some examples, the displayed images (e.g., a series of screen shots from the display device) may be provided to an internal or external processing device. The power Doppler colour-bar included in each image may be used to threshold and segment power Doppler data within the ultrasound image cone. That is, any pixels in the image shown to have an image value (e.g., expressed as an RGB data set) which belong to the set of values included in the power Doppler colour-bar may be segmented as power Doppler pixels. In one example, the segmentation may be implemented by binarizing power Doppler values in the image cone—e.g., 1 for any pixel with power Doppler values, and 0 for any pixels not having power Doppler values. The “power Doppler area percentage”—i.e. the percentage of the total number of pixels within the image cone which are determined to be “power Doppler pixels”—may be calculated for each image frame. The plurality of power Doppler area percentages values may be recorded over the duration of the acquisition of data.

The power Doppler area percentage values may be filtered in block 418 for example by use of a rolling window so as to smooth the data. For instance, the power Doppler area percentage values may be averaged by a rolling window, which may take an average such as a mean average of values in the window. The mean rolling window may be of, e.g., 10 samples in window width which provided acceptable results, when assessed manually and smoothed out any irregular changes caused by transducer movement or due to the breathing of the patient. The 10 samples window width is an example width, provided for typical image frame rates, such as approximately but not necessarily 60 frames per second. Other numerical techniques for smoothing and filtering the data can also be performed to avoid noise causing the algorithm to provide a value that does not correspond to a true sub-noise gain value.

The decibel level of the power Doppler gain displayed on the screen may be extracted from the image data by comparison of the image data to a data dictionary of known alpha-numeric characters (digits, minus sign and decimal point). In one example implementation, this is done by comparing the dictionary of patches against the decibel values displayed as text in the image and using a metric of image similarity (normalised cross-correlation) above a set value (e.g. >0.85) to indicate what numeric values were displayed. The numeric values corresponding to the gain level are then stored on per-frame basis in a memory location accessible by the processing device where the SNG setting module is installed. This data may further be filtered by using a rolling-median window (e.g., of 3 samples) to remove any outliers.

Following the percentage determination at block 404, optimisation algorithms may be applied to the percentage data, to determine an optimal level of gain. The optimal level is used here in a general sense here to mean the level at which the maximum, or at least an acceptable level, of “true” power Doppler data is displayed, prior to the appearance of noise bloom. This optimal level may be determined through blocks 406 and 408.

At block 406 statistical analysis is applied to the series of power Doppler data percentages 403 to determine a breakpoint in the series of power Doppler data percentages which corresponds with a noise bloom. At block 408 a sub-noise gain level is determined based upon a power Doppler gain level corresponding to the breakpoint or based upon a power Doppler gain level corresponding to a point calculated from the breakpoint.

Block 406 thus involves identifying a breakpoint in the series of power Doppler data percentages, at which a noise bloom effect is observed. Block 408 involves determining an optimal sub noise gain level based on the position of the breakpoint.

The series of ultrasound data sets may correspond to a series of ultrasound image frames taken in succession. Therefore, determining the optimal sub noise gain level at block 408 may comprise selecting an image frame, or frame position in the series, at which the image has optimal gain. In some examples, this may comprise selecting a frame corresponding to the breakpoint. In other examples, this may comprise selecting a frame occurring prior to the breakpoint (i.e. at a lower gain level than the breakpoint). For instance, in some examples, the optimal frame position may be determined by applying an offset amount, such as a predetermined number of fames, to the breakpoint.

The optimal sub-noise gain 421 may be determined by looking up the power Doppler gain level applied to the image data at the identified “optimal frame location”. In one example, the frame position corresponding to the optimal gain level is determined by subtracting an offset amount from the breakpoint position determined from the statistical analysis. For instance, if the statistical analysis identifies frame 300 (at a frame rate of 60 frames per second) as showing the bloom effect, and the offset amount applied by the algorithm is 25 frames, then the algorithm will determine the optimal gain level to be that applied at frame 275. The offset amount of 25 frames (or 25 samples) is an example only. Other offset amounts may be used to determine the frame or sample position which corresponds with the optimal gain level.

The identification of the breakpoint may comprise applying statistical algorithm(s) to the series of power Doppler data percentage values 403. The series of values 403 may be analysed as a function of the time elapsed since the start of scanning (determined by frame rate and the “position” of the frame in the sequence of image frames), or as a function of the frame “position”.

At block 406, various statistical methods may be applied to determine the breakpoint corresponding with the noise bloom. In some examples, the statistical analysis comprises determining an inflection point in the series of power Doppler data percentages. In some examples, the statistical analysis comprises a piece-wise separation of the series of power Doppler data percentages into two or more regression models based on a known number of segments.

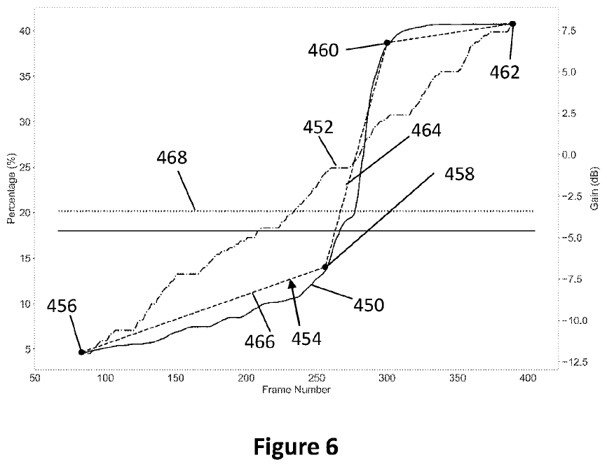

FIG. 6 shows example plots of “power Doppler data percentage” and “gain level” vs frame position (“frame number”). The solid line 450 shows the percentages of power Doppler data in the image frames. The dot-dash line 452 shows the gain levels applied at the time of each of the image frames. Therefore the solid line 450 represents the series of power Doppler data percentages and the dot-dash line 452 the gain levels of the series of ultrasound data sets. A linear regression may be applied to the power Doppler data percentages in order to determine one or more breakpoints and a breakpoint corresponding to a noise bloom may then be determined. The breakpoint corresponding to the noise bloom may be determined to be a breakpoint after which a higher rate of increase of power Doppler data percentages occurs compared to other breakpoints. An example is given below.

The dashed line 454 is an example of a piece-wise linear regression applied to the power Doppler data percentages. In this example, the regression is of a type known to have 3 segments, but with unknown positions of the segment endpoints or “breakpoints” for the percentage data curve 450. The optimal positions of these breakpoints can be determined as follows. For any given set of “b” breakpoint locations (where the number of breakpoints is n_{b}), a least squares fit can be performed which solves for the β parameters that minimize the “sum-of-square” error of the residuals (for example, where Aβ=y and A is the n×n_{b} regression matrix, β is the vector (n_{b}×1) of unknown parameters, and y is the vector (n×1) of y data points).

In the case of the three-segment regression 454, n_{b} is set to 4 as the three segments are defined between the four breakpoints. The sum-of-square of the residuals (SSR) can be represented as a function dependent on the breakpoint locations SSR(b). The regression algorithm can be initiated with the assumption that the first breakpoint b₁ is at position x₁ (i.e. the smallest x value), and the last breakpoint b_{nb} is at position Xₙ (i.e. the largest x). An optimization problem may be formulated to find the breakpoint locations that minimize the overall sum-of-square of the residuals. The statistical algorithms included thus formulates this problem as a global function. The algorithms may implement a differential evolution strategy that solves this global function.

The piece-wise linear approximation 454 may be defined by a number of points: including a starting point 456 corresponding to a start of the series of image frames, a first turning point 458 which corresponds to a first inflection point in the percentage data 450, a second turning point 460 which corresponds to a second inflection point in the percentage data 450, and an end point 462 corresponding to an end of the series of image frames. The positions of these points may be determined using the method described in the previous paragraphs. As can be seen, the linear segment 464 between the two turning points 458, 460 appears to show a much higher rate of increase in the percentage data, compared with the linear segment 466 between the starting point 456 and the first turning point 458. The first turning point 458 may therefore be identified as the breakpoint where the noise bloom has occurred.

While a piece-wise linear regression has been described above, it will be appreciated than in other examples different types of statistical analysis could be used to determine a breakpoint in the series of power Doppler data percentages which corresponds with a noise bloom. For example, a statistical analysis which comprises which regresses the distribution of power Doppler percentage data values against gain may be used. The statistical analysis is not limited to a linear piecewise regression model, as other models such as polynomial or spline regression models may be used. In the case of a polynomial or spine regression model, the model may be assigned a set number of degrees of freedom. In this case the model will provide a regression which has a number of turning points and the break point corresponding to the noise bloom will be the break point with the largest gradient. The method may comprise finding the turning point having the largest gradient, e.g. using automated methods for determining the gradient, and determining that the turning point having the largest gradient is the break point corresponding to the noise bloom.

In the example of FIG. 6, the optimal gain position is identified by subtracting an offset amount from the position of the first turning point 458. The first turning point 458 calculated corresponds to approximately the 260^{th} frame. The optimal frame position is taken at a 30 frame offset and therefore corresponds to approximately the 230^{th} frame. The gain at the 230^{th} frame (vertical line 470) is shown by the dotted horizontal line 468, at approximately −2.5 dB. This optimal gain level is then used to set the sub-noise gain setting. In other examples, a different number of frames could be used for the offset. In some examples, a gain value interpolated between frames may be used.

Referring back to the apparatus shown in FIG. 3, block 404 of the method of FIG. 4 may be implemented by a percentage determination module 342 of the module 340 shown in FIG. 3. Blocks 406 and 408 of the method of FIG. 4 may be implemented by an optimal gain determination module 344 of the module 340 shown in FIG. 3. The optimal gain determination module 344 may identify the break point, at which the aforementioned noise “bloom” occurs, and determine the optimal gain level using information relating to the identified breakpoint. The position of the breakpoint can be expressed as the position of the image frame, or in other examples may be expressed as time.

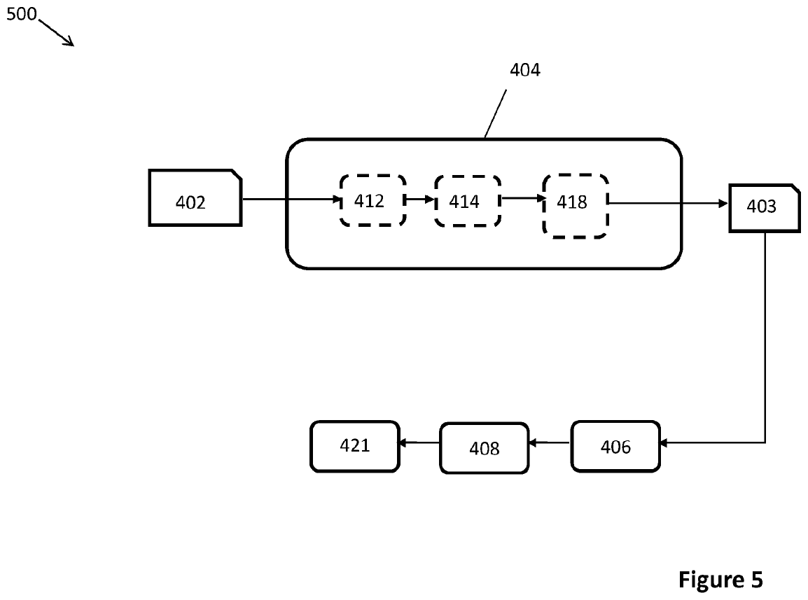

FIG. 5 schematically depicts a further example method 500 for determining the sub-noise gain level, in which like reference numerals denote like processes as in FIG. 4. In the example of FIG. 5, the processing module which implements the method has access to the numerical data from the ultrasound machine. Therefore the method makes use of ultrasound data which is directly available from the ultrasound machine, rather analysing images output by the ultrasound machine or displayed on the display of the ultrasound machine. For instance, the processing module may have access to data such as: the number of pixels showing power Doppler data, the number of pixels showing B-mode data, the percentage of the data that corresponds with power Doppler data in the data set corresponding to each ultrasound slice, the gain level corresponding to a particular ultrasound slice, etc. These may be directly read from the ultrasound machine at block 412, e.g., from data buffers in the processing device of the machine. Simple arithmetic calculations, if required, may then be performed to calculate the percentage at block 414. The power Doppler data percentages may then be filtered or averaged at block 418. Blocks 402, 403, 406, 408 and 421 may be as described for FIG. 4.

The method of FIGS. 4 and 5 may be implemented by a processing module 340 for determining the sub-noise gain. The processing module 340 may be provided as part of the ultrasound machine 300 as shown in FIG. 3. The ultrasound machine 300 may include a transducer 310 for acquiring ultrasound data from the scanned tissue 100 and transmitting the ultrasound data to the controller 320 of the ultrasound machine. The received ultrasound data may be used to generate images to be displayed by a screen or other display device 330 of the ultrasound machine.

The module 340 may obtain the ultrasound data directly from the ultrasound machine (arrow 504), or acquire the screen data from the display (arrow 506). In some examples, the module 340 may be configured to get ultrasound data from both sources (if the data from the ultrasound is made available), so as to allow a selection depending on the available data source(s). The received ultrasound data is used by the percentage determination module 342 to determine the power Doppler data percentage level values. The percentage values, as a function of time or image frame position, may be provided to the optimal gain determination module 344, which may be configure to perform breakpoint determination to determine where in the series of images (or when during the acquisition time) the breakpoint in the ultrasound data occurs, and then identify the optimal gain level from the identified breakpoint or a position calculated from the breakpoint.

Depending on the embodiment, the optimal gain level 350 may be provided to the communicated to the sonographer, e.g. by being output on a display device 360 receiving data from the processing module 340. The sonographer can then adjust the SNG setting to the calculated optimal gain level, and restart or resume the scanning process.

The optimal gain level may instead be directly provided to or within the controller 340 of the ultrasound machine (dashed arrow 352) if the processing module 340 is provided internal to the ultrasound machine, or is otherwise in communication with the ultrasound machine. The optimal level can then be displayed on the display 330 of or connected to the ultrasound machine. In some examples, the gain level may be automatically adjusted by the controller 320 of the ultrasound machine. The processing module 340 may be provided as an internal module within the controller 320 of the ultrasound machine.

As discussed earlier, the aforementioned process for identifying the optimal gain level may be included as part of a set up process, to determine the optimal gain level, and use the optimal gain level to acquire the clinical data to be interpreted. As the point at which the “bloom” effect will occur may depend on physiological factors (such as adiposity or tissue homogeneity), when scanning a large area or volume, as discussed above, the optimal gain level may be recalibrated at various sub-volumes corresponding to different depths across the area or volume of interest.

In some examples, the optimal sub-noise gain level may be determined for a first sub-volume of the tissue volume of interest, and a power Doppler ultrasound performed at the first sub-volume of the tissue volume of interest at the sub-noise gain level determined for the first sub-volume. The sub-noise gain level may be re-calculated for one or more other sub-volumes of the tissue volume of interest and power Doppler ultrasound scan(s) may be performed at the one or more other sub-volumes of the tissue volume of interest at the re-calculated sub-noise gain level(s).

The ultrasound processing, sub noise gain level determination and/or automated machine settings described herein may be performed in software, firmware, hardware, or a combination thereof. In the examples above, the processes which implement the gain level optimisation may be provided within the same processing device, or on different processing devices.

Variations and modifications may be made to the parts previously described without departing from the spirit or ambit of the disclosure.

Furthermore, except where explicitly indicated this is not the case or where logic dictates otherwise, features and steps disclosed in respect of one example or embodiment may be used in the other examples or embodiments disclosed herein.

1. A method for determining a sub-noise gain level for performing a power Doppler ultrasound scan of a tissue volume of interest, the method comprising:
receiving a series of ultrasound data sets, each ultrasound data set corresponding to a same tissue volume of interest, each ultrasound data set having been acquired at a different respective power Doppler gain level;
for each ultrasound data set, determining a power Doppler data percentage, being a percentage of data in the ultrasound data set which corresponds to power Doppler data, wherein the determined power Doppler data percentages form a series of power Doppler data percentages corresponding to the series of ultrasound data sets;
applying statistical analysis to the series of power Doppler data percentages, to determine a breakpoint in the series of power Doppler data percentages which corresponds with a noise bloom; and
determining a sub-noise gain level based upon a power Doppler gain level corresponding to the breakpoint or based upon a power Doppler gain level corresponding to a point calculated from the breakpoint.

2. The method of claim 1, wherein said determining of the sub-noise gain level comprises looking up a power Doppler gain level at which an ultrasound data set corresponding to the breakpoint was acquired.

3. The method of claim 1, wherein said determining of the sub-noise gain level comprises determining a power Doppler gain level of a point calculated from the breakpoint.

4. The method of claim 3, wherein the point calculated from the breakpoint is offset by a predetermined amount from the breakpoint.

5. The method of claim 1, further comprising filtering the power Doppler data percentages with a rolling window.

6. The method of claim 5, wherein said filtering of the power Doppler data percentages with the rolling window comprises averaging the power Doppler data percentages over a rolling window.

7. The method of claim 1, wherein each ultrasound data set includes an ultrasound image comprising an ultrasound cone portion, and the percentage Doppler data percentage for each ultrasound data set is determined by segmenting pixels in the ultrasound cone portion of the image which have power Doppler data values.

8. The method of claim 7, wherein the ultrasound image further includes a power Doppler colour bar portion, wherein the segmented pixels have colour values which are also found in any of the pixels in the power Doppler colour bar portion.

9. The method of claim 1, wherein the statistical analysis comprises determining an inflection point in the series of power Doppler data percentages.

10. The method of claim 1, wherein the statistical analysis comprises a piece-wise separation of the series of power Doppler data percentages into two or more regression models based on a known number of segments.

11. The method of claim 1, further comprising initialising an ultrasound machine by controlling the ultrasound machine to generate the series of power Doppler ultrasound data sets by performing a plurality of power Doppler ultrasound scans at a plurality of different power Doppler gain levels at a same location on the tissue volume of interest.

12. The method of claim 1, wherein the sub-noise gain level is determined for the tissue volume of interest, and wherein the method further comprises performing a power Doppler ultrasound scan of the tissue volume of interest at the determined sub-noise gain level.

13. The method of claim 1, wherein the tissue volume of interest is divided into a plurality of sub-volumes of interest based on depth and wherein the method comprises determining a sub-noise gain level for each sub-volume of interest, wherein each sub-volume of interest corresponds to a different depth.

14. A non-transitory machine readable storage medium storing instructions executable by a processor to:
receive a series of ultrasound data sets, each ultrasound data set corresponding to a same tissue volume of interest, each ultrasound data set having been acquired at a different respective power Doppler gain level;
for each ultrasound data set, determine a power Doppler data percentage, being a percentage of data in the ultrasound data set which corresponds to power Doppler data,
wherein the determined power Doppler data percentages form a series of power Doppler data percentages corresponding to the series of ultrasound data sets:
apply statistical analysis to the series of power Doppler data percentages, to determine a breakpoint in the series of power Doppler data percentages which corresponds with a noise bloom; and
determine a sub-noise gain level based upon a power Doppler gain level corresponding to the breakpoint or based upon a power Doppler gain level corresponding to a point calculated from the breakpoint.

15. An ultrasound apparatus, comprising:
a transducer for sending and receiving an ultrasound signal;
a display for displaying ultrasound data;
a controller for controlling the transducer to perform a power Doppler ultrasound scan; and
a sub-noise gain module configured to determine a sub-noise gain level for performing power Doppler ultrasound on a tissue volume of interest by: by
receiving a series of ultrasound data sets, each ultrasound data set corresponding to a same tissue volume of interest, each ultrasound data set having been acquired at a different respective power Doppler gain level:
for each ultrasound data set. determining a power Doppler data percentage, being a percentage of data in the ultrasound data set which corresponds to power Doppler data, wherein the determined power Doppler data percentages form a series of power Doppler data percentages corresponding to the series of ultrasound data sets;
applying statistical analysis to the series of power Doppler data percentages, to determine a breakpoint in the series of power Doppler data percentages which corresponds with a noise bloom; and
determining a sub-noise gain level based upon a power Doppler gain level corresponding to the breakpoint or based upon a power Doppler gain level corresponding to a point calculated from the breakpoint:
wherein the sub-noise gain module is implemented by a processor.

16. The ultrasound apparatus of claim 15, wherein the sub-noise gain module is configured to determine the sub-noise gain level by determining a power Doppler gain level of a point calculated from the breakpoint.

17. The ultrasound apparatus of claim 15, wherein the statistical analysis comprises a piece-wise separation of the series of power Doppler data percentages into two or more regression models based on a known number of segments.

18. The non-transitory machine readable storage medium of claim 14, wherein the instructions include instructions to determine the sub-noise gain level by looking up a power Doppler gain level at which an ultrasound data set corresponding to the breakpoint was acquired.

19. The non-transitory machine readable storage medium of claim 14, wherein the instructions include instructions to determine the sub-noise gain level by determining a power Doppler gain level of a point calculated from the breakpoint.

20. The non-transitory machine readable storage medium of claim 14, wherein the statistical analysis comprises a piece-wise separation of the series of power Doppler data percentages into two or more regression models based on a known number of segments.
